(19) United States
(12) Patent Application Publication
(10) Pub. No.: US 2023/0062781 A1
(43) Pub. Date: Mar. 2, 2023

(54) METHODS AND SYSTEMS FOR IMPLEMENTING AND USING DIGITAL IMAGING AND COMMUNICATIONS IN MEDICINE (DICOM) STRUCTURED REPORTING (SR) OBJECT CONSOLIDATION

(71) Applicant: GE Precision Healthcare LLC, Wauwatosa, WI (US)

(72) Inventors: Tracy Hunter, Waukesha, WI (US); Peter Wlczek, Wartenberg (DE); Christian Walheim, Freiburg (DE); Steven Nichols, Okauchee, WI (US)

(21) Appl. No.: 17/459,542

(22) Filed: Aug. 27, 2021

(51) Int. Cl. G16H 30/20; G16H 30/40; G16H 15/00; G16H 50/20; G16H 50/70; G06T 7/00

(52) U.S. Cl. CPC G16H 30/20; G16H 30/40; G16H 15/00; G16H 50/20; G16H 50/70; G06T 7/0014; A61B 8/00

(57) ABSTRACT

Systems and methods for implementing and using digital imaging and communications in medicine (DICOM) structured reporting (SR) object consolidation. The consolidation process may be applied to a plurality of objects generated based on a same medical imaging data. The consolidation process includes assessing each object of the plurality of objects, with the assessing including determining whether the object is a parent of another object in the plurality of objects, and discarding the object when it is a parent of another object. A composite object is then generated based on the plurality of objects, with the generating including, when only one object remains after the assessing, copying that object into the consolidated object; otherwise when a plurality of remaining objects remains after the assessing, processing the remaining objects, in sequence from newest to oldest, with the processing including, for copying each data element in each remaining object into the composite object.

### FIELD

Aspects of the present disclosure relate to medical imaging solutions. More specifically, certain embodiments relate to methods and systems for implementing and using digital imaging and communications in medicine (DICOM) structured reporting (SR) object consolidation.

### BACKGROUND

Various medical imaging techniques may be used, such as in imaging organs and soft tissues in a human body. Examples of medical imaging techniques include ultrasound imaging, computed tomography (CT) scans, magnetic resonance imaging (MRI), etc. The manner by which images are generated during medical imaging depends on the particular technique.

For example, ultrasound imaging uses real time, non-invasive high frequency sound waves to produce ultrasound images, typically of organs, tissues, objects (e.g., fetus) inside the human body. Images produced or generated during medical imaging may be two-dimensional (2D), three-dimensional (3D), and/or four-dimensional (4D) images (essentially real-time/continuous 3D images). During medical imaging, imaging datasets (including, e.g., volumetric imaging datasets during 3D/4D imaging) are acquired and used in generating and rendering corresponding images (e.g., via a display) in real-time.

In some instances, there may be a need to manage imaging data generated during and/or based on medical imaging, in particular with respect to managing analysis and assessment of the imaging data, particularly when conducted by various users. Such scenarios may pose certain challenges, particularly with respect ensuring reliability and integrity of the imaging data and/or information obtained based thereon. Limitations and disadvantages of conventional approaches, if any existed, for handling such situations will become apparent to one of skill in the art, through comparison of such approaches with some aspects of the present disclosure, as set forth in the remainder of the present application with reference to the drawings.

### BRIEF SUMMARY

System and methods are provided for implementing and using digital imaging and communications in medicine (DICOM) structured reporting (SR) object consolidation, substantially as shown in and/or described in connection with at least one of the figures, as set forth more completely in the claims.

These and other advantages, aspects and novel features of the present disclosure, as well as details of one or more illustrated example embodiments thereof, will be more fully understood from the following description and drawings.

### BRIEF DESCRIPTION OF THE DRAWINGS

FIG. 1 is a block diagram illustrating an example medical imaging arrangement.

FIG. 2 is a block diagram illustrating an example ultrasound imaging system.

FIG. 3 is a block diagram illustrating an example use scenario for consolidating multiple digital imaging and communications in medicine (DICOM) structured reporting (SR) objects.

FIG. 4 illustrates a flowchart of an example process for digital imaging and communications in medicine (DICOM) structured reporting (SR) object consolidation.

### DETAILED DESCRIPTION

Certain implementations in accordance with the present disclosure may be directed to implementing and using digital imaging and communications in medicine (DICOM) structured reporting (SR) object consolidation. In particular, the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” as used in the context of ultrasound imaging is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

In addition, as used herein, the phrase “pixel” also includes embodiments where the data is represented by a “voxel.” Thus, both the terms “pixel” and “voxel” may be used interchangeably throughout this document.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC, or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. In addition, forming of images may be performed using channel combinations that may originate from more than one transmit event (e.g., synthetic aperture techniques).

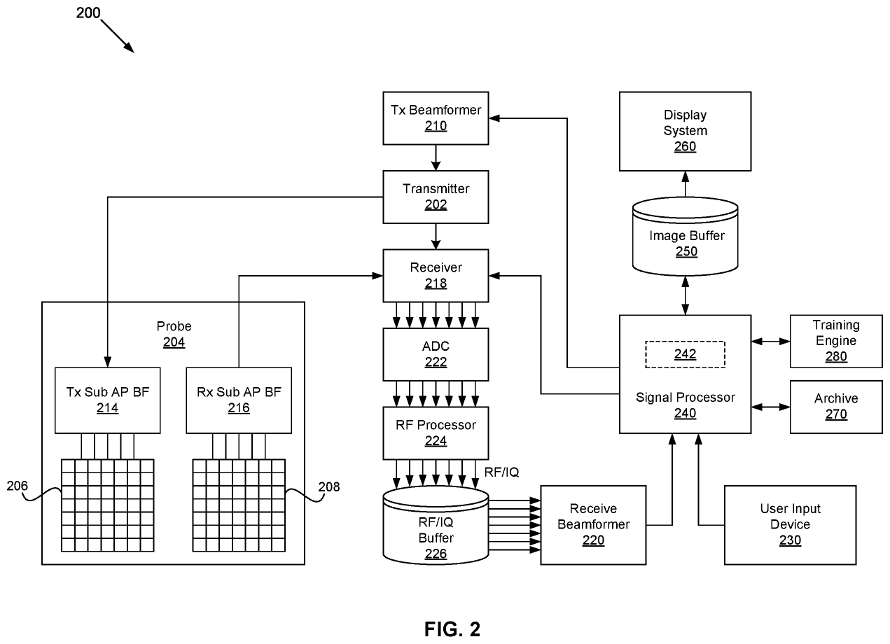

In various embodiments, processing to form images is performed in software, firmware, hardware, or a combination thereof. The processing may include use of beamforming. One example implementation of an ultrasound system having a software beamformer architecture formed in accordance with various embodiments as illustrated in FIG. 2.

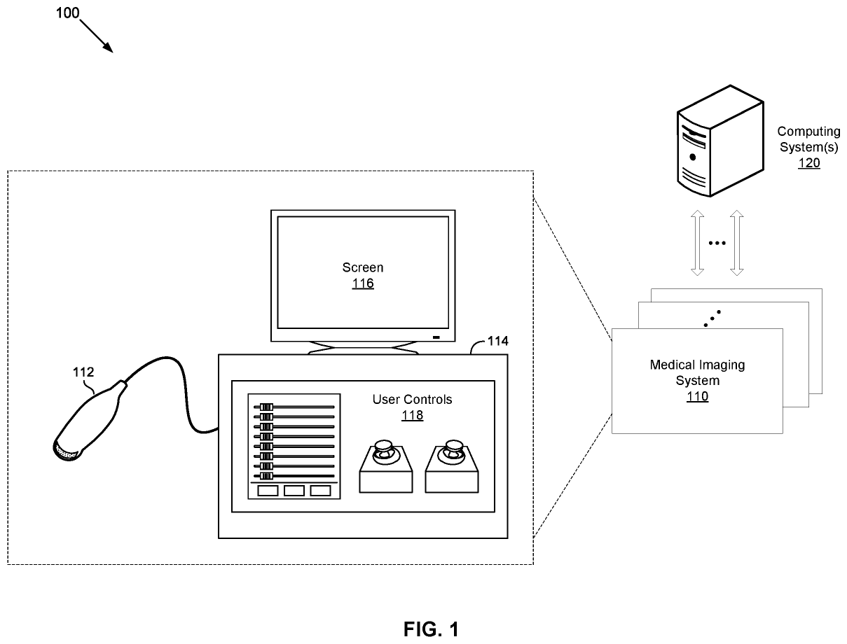

FIG. 1 is a block diagram illustrating an example medical imaging arrangement. Shown in FIG. 1 is an example medical imaging arrangement 100 that comprises one or more medical imaging systems 110 and one or more computing systems 120. The medical imaging arrangement 100 (including various elements thereof) may be configured to support implementing and using digital imaging and communications in medicine (DICOM) structured reporting (SR) object consolidation in accordance with the present disclosure.

The medical imaging system 110 comprise suitable hardware, software, or a combination thereof, for supporting medical imaging—that is enabling obtaining data used in generating and/or rendering images during medical imaging exams. Examples of medical imaging include ultrasound imaging, computed tomography (CT) scans, magnetic resonance imaging (MRI), etc. This may entail capturing of particular type of data, in particular manner, which may in turn be used in generating data for the images. For example, the medical imaging system 110 may be an ultrasound imaging system, configured for generating and/or rendering ultrasound images. An example implementation of an ultrasound system, which may correspond to the medical imaging system 110, is described in more detail with respect to FIG. 2.

As shown in FIG. 1, the medical imaging system 110 may comprise a scanner device 112, which may be portable and movable, and a display/control unit 114. The scanner device 112 may be configured for generating and/or capturing particular type of imaging signals (and/or data corresponding thereto), such as by being moved over a patient's body (or part thereof), and may comprise suitable circuitry for performing and/or supporting such functions. The scanner device 112 may be an ultrasound probe, MRI scanner, CT scanner, or any suitable imaging device. For example, where the medical imaging system 110 is an ultrasound system, the scanner device 112 may emit ultrasound signals and capture echo ultrasound images.

The display/control unit 114 may be configured for displaying images (e.g., via a screen 116). In some instances, the display/control unit 114 may further be configured for generating the displayed images, at least partly. Further, the display/control unit 114 may also support user input/output. For example, the display/control unit 114 may provide (e.g., via the screen 116), in addition to the images, user feedback (e.g., information relating to the system, functions thereof, settings thereof, etc.). The display/control unit 114 may also support user input (e.g., via user controls 118), such as to allow controlling of the medical imaging. The user input may be directed to controlling display of images, selecting settings, specifying user preferences, requesting feedback, etc.

In some implementations, the medical imaging arrangement 100 may also incorporate additional and dedicated computing resources, such as the one or more computing systems 120. In this regard, each computing system 120 may comprise suitable circuitry, interfaces, logic, and/or code for processing, storing, and/or communication data. The computing system 120 may be dedicated equipment configured particularly for use in conjunction with medical imaging, or it may be a general purpose computing system (e.g., personal computer, server, etc.) set up and/or configured to perform the operations described hereinafter with respect to the computing system 120. The computing system 120 may be configured to support operations of the medical imaging systems 110, as described below. In this regard, various functions and/or operations may be offloaded from the imaging systems. This may be done to streamline and/or centralize certain aspects of the processing, to reduce cost—e.g., by obviating the need to increase processing resources in the imaging systems.

The computing systems 120 may be set up and/or arranged for use in different ways. For example, in some implementations a single computing system 120 may be used; in other implementations multiple computing systems 120, either configured to work together (e.g., based on distributed-processing configuration), or separately, with each computing system 120 being configured to handle particular aspects and/or functions, and/or to process data only for particular medical imaging systems 110. Further, in some implementations, the computing systems 120 may be local (e.g., co-located with one or more medical imaging systems 110, such within the same facility and/or same local network); in other implementations, the computing systems 120 may be remote and thus can only be accessed via remote connections (e.g., via the Internet or other available remote access techniques). In a particular implementation, the computing systems 120 may be configured in cloud-based manner, and may be accessed and/or used in substantially similar way that other cloud-based systems are accessed and used.

Once data is generated and/or configured in the computing system 120, the data may be copied and/or loaded into the medical imaging systems 110. This may be done in different ways. For example, the data may be loaded via directed connections or links between the medical imaging systems 110 and the computing system 120. In this regard, communications between the different elements in the medical imaging arrangement 100 may be done using available wired and/or wireless connections, and/or in accordance any suitable communication (and/or networking) standards or protocols. Alternatively, or additionally, the data may be loaded into the medical imaging systems 110 indirectly. For example, the data may be stored into suitable machine readable media (e.g., flash card, etc.), which are then used to load the data into the medical imaging systems 110 (on-site, such as by users of the systems (e.g., imaging clinicians) or authorized personnel), or the data may be downloaded into local communication-capable electronic devices (e.g., laptops, etc.), which are then used on-site (e.g., by users of the systems or authorized personnel) to upload the data into the medical imaging systems 110, via direct connections (e.g., USB connector, etc.).

In operation, the medical imaging system 110 may be used in generating and presenting (e.g., rendering or displaying) images during medical exams, and/or in supporting user input/output in conjunction therewith. The images may be 2D, 3D, and/or 4D images. The particular operations or functions performed in the medical imaging system 110 to facilitate the generating and/or presenting of images depends on the type of system—that is, the manner by which the data corresponding to the images is obtained and/or generated. For example, in computed tomography (CT) scans based imaging, the data is based on emitted and captured x-rays signals. In ultrasound imaging, the data is based on emitted and echo ultrasound signals, as described in more detail with respect to FIG. 2.

In various implementations in accordance with the present disclosure, medical imaging systems and/or architectures (e.g., the medical imaging system 110 and/or the medical imaging arrangement 100 as a whole) may be configured to support enhanced solutions for storage and management of medical imaging data. In particular, medical imaging solutions may be configured and/or modified to incorporate enhanced Digital Imaging and Communications in Medicine (DICOM) based functions, such as Structured Reports (SR) object consolidation. A consolidation scheme/methodology in accordance with the present disclosure may be used by any application that consumes DICOM SR objects like reporting and analytics packages.

In this regard, DICOM is an international standard for the communication and management of medical imaging information and related data. The DICOM standard describes how medical data may be represent in files and how it may be exchanged—e.g., defining both the format of the files and network transfer protocols. In this regard, the DICOM standard defines various structures for use in conjunction with the storage, management and communication of imaging data. For example, The DICOM 3.0 standard defines several object types called Structured Reports (SR) objects, which may be used to facilitate exchange medical findings between software applications. As used in the standard, an SR (report) does not necessarily mean report in the “clinical” sense; rather, it may merely be or correspond to an observation based on the corresponding imaging data. The SRs are created and accompany the corresponding image files, including information relating to these image files or images associated therewith (e.g., measurements, information relating to imaged structures or features therein, etc.).

When multiple SR objects exist for a study, applications that consume these SR object sets have a challenge consolidating the content. In this regard, there may be different types of SR objects—e.g., SRs may be of two main different varieties: 1) “final” SRs, which may contain the “final” information relating to the image files; and 2) “intermediate” or “incomplete” SRs. An “intermediate” or “incomplete” SR documents details of observation. Thus may be problematic, however, as there may be multiple observations—e.g., in the context of heart imaging, there may be anatomy related observations, blood flow related observations, etc. Then they are calculations that may be made based on these observation to come up with conclusions. Further, different persons (clinician, doctors, etc.) may review images and the related measurements and may make new measurements, thus resulting in new calculations.

Thus, in some instances multiple reports maybe generated and may need to be digested. The DICOM 3.0 standard does not provide a mechanism for consolidating multiple SR objects associated with a particular study—e.g., all the different intermediate/incomplete SR objects. In this regard, existing solutions typically focus on creating the findings (e.g., measurements) and SR objects, with little (if any) functions for enabling consumers of the data collate and resolve conflicting data elements from multiple SR objects. Solutions in accordance with the present disclosure address such issues, by incorporation mechanisms for consolidating multiple SR objects—e.g., automatically consolidates the data while resolving data conflicts.

These mechanisms may be configured to, e.g., automatically identify anomalies and/or differences between the SR objects, and to resolve/reconcile these anomalies and/or differences. This may be done, for example, by use of a consolidator module, which may be deployed and used to manage multiple SR objects when they may be created. Such consolidator may be deployed adaptively—e.g., in the medical imaging equipment, in a local dedicated system, or even in remote entity (e.g., cloud-based system); or alternatively, may be deployed in distributed manner, with different functions or elements thereof being deployed in and/or performed in different components within the imaging environment. In some instances, advanced processing techniques may be used to further enhance handling of the multiple SR objects. For example, in some example implementations, artificial intelligence (AI) based learning mode may also be used, such as to recognize common manual anomaly reconciliations to make them automatic.

Solutions in accordance with the present disclosure offer various technical and commercial benefits over existing solutions. In this regard, having an independent application to prepare the SR data for consumption has the following benefits. For example, use of such dedicated consolidation function relieves the need for DICOM based applications from needing subject matter expertise on DICOM SR formatting. Also, use of such dedicated consolidation function may allow for avoiding errors that may arise from data conflicts and anomalies. Further, use of such dedicated consolidation function may allow for resolving differences between vendors that create DICOM SR objects.

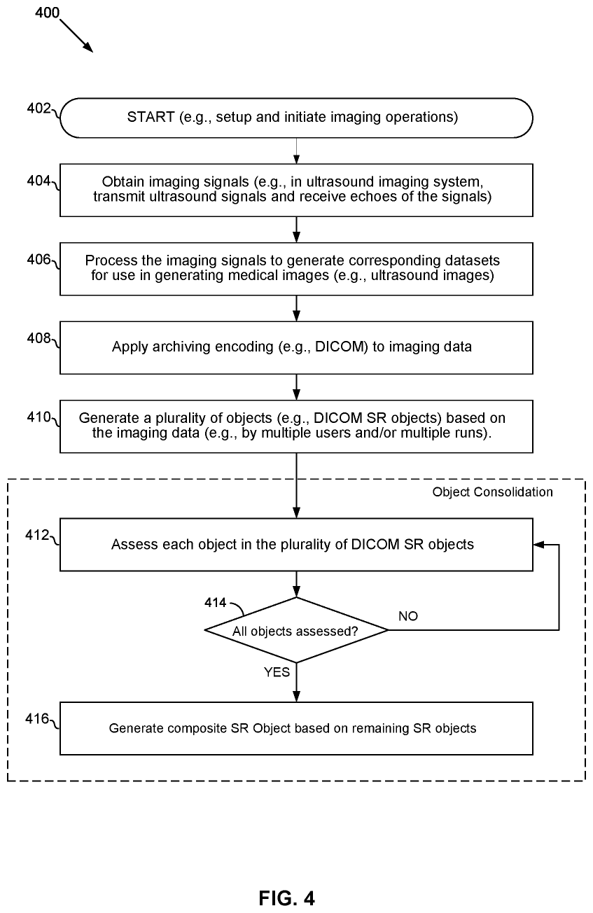

Example implementations and use cases/scenarios based on solutions in accordance with the present disclosure are described in more detail below, particularly in conjunction with the example use case scenario illustrated in FIG. 4.

FIG. 2 is a block diagram illustrating an example ultrasound imaging system. Shown in FIG. 2 is an ultrasound imaging system 200, which may be configured to support implementing and using digital imaging and communications in medicine (DICOM) structured reporting (SR) object consolidation in accordance with the present disclosure.

The ultrasound imaging system 200 may be configured for providing ultrasound imaging, and as such may comprise suitable circuitry, interfaces, logic, and/or code for performing and/or supporting ultrasound imaging related functions. The ultrasound imaging system 200 may correspond to the medical imaging system 110 of FIG. 1. The ultrasound imaging system 200 comprises, for example, a transmitter 202, an ultrasound probe 204, a transmit beamformer 210, a receiver 218, a receive beamformer 220, a RF processor 224, a RF/IQ buffer 226, a user input module 230, a signal processor 240, an image buffer 250, a display system 260, an archive 270, and a training engine 280.

The transmitter 202 may comprise suitable circuitry, interfaces, logic, and/or code that may be operable to drive an ultrasound probe 204. The ultrasound probe 204 may comprise a two dimensional (2D) array of piezoelectric elements. The ultrasound probe 204 may comprise a group of transmit transducer elements 206 and a group of receive transducer elements 208, that normally constitute the same elements. In certain embodiment, the ultrasound probe 204 may be operable to acquire ultrasound image data covering at least a substantial portion of an anatomy, such as the heart, a blood vessel, or any suitable anatomical structure.

The transmit beamformer 210 may comprise suitable circuitry, interfaces, logic, and/or code that may be operable to control the transmitter 202 which, through a transmit sub-aperture beamformer 214, drives the group of transmit transducer elements 206 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 208.

The group of receive transducer elements 208 in the ultrasound probe 204 may be operable to convert the received echoes into analog signals, undergo sub-aperture beamforming by a receive sub-aperture beamformer 216 and are then communicated to a receiver 218. The receiver 218 may comprise suitable circuitry, interfaces, logic, and/or code that may be operable to receive the signals from the receive sub-aperture beamformer 216. The analog signals may be communicated to one or more of the plurality of A/D converters 222.

The plurality of A/D converters 222 may comprise suitable circuitry, interfaces, logic, and/or code that may be operable to convert the analog signals from the receiver 218 to corresponding digital signals. The plurality of A/D converters 222 are disposed between the receiver 218 and the RF processor 224. Notwithstanding, the disclosure is not limited in this regard. Accordingly, in some embodiments, the plurality of A/D converters 222 may be integrated within the receiver 218.

The RF processor 224 may comprise suitable circuitry, interfaces, logic, and/or code that may be operable to demodulate the digital signals output by the plurality of A/D converters 222. In accordance with an embodiment, the RF processor 224 may comprise a complex demodulator (not shown) that is operable to demodulate the digital signals to form I/Q data pairs that are representative of the corresponding echo signals. The RF or I/Q signal data may then be communicated to an RF/IQ buffer 226. The RF/IQ buffer 226 may comprise suitable circuitry, interfaces, logic, and/or code that may be operable to provide temporary storage of the RF or I/Q signal data, which is generated by the RF processor 224.

The receive beamformer 220 may comprise suitable circuitry, interfaces, logic, and/or code that may be operable to perform digital beamforming processing to, for example, sum the delayed channel signals received from RF processor 224 via the RF/IQ buffer 226 and output a beam summed signal. The resulting processed information may be the beam summed signal that is output from the receive beamformer 220 and communicated to the signal processor 240. In accordance with some embodiments, the receiver 218, the plurality of A/D converters 222, the RF processor 224, and the beamformer 220 may be integrated into a single beamformer, which may be digital. In various embodiments, the ultrasound imaging system 200 comprises a plurality of receive beamformers 220.

The user input device 230 may be utilized to input patient data, scan parameters, settings, select protocols and/or templates, interact with an artificial intelligence segmentation processor to select tracking targets, and the like. In an example embodiment, the user input device 230 may be operable to configure, manage and/or control operation of one or more components and/or modules in the ultrasound imaging system 200. In this regard, the user input device 230 may be operable to configure, manage and/or control operation of the transmitter 202, the ultrasound probe 204, the transmit beamformer 210, the receiver 218, the receive beamformer 220, the RF processor 224, the RF/IQ buffer 226, the user input device 230, the signal processor 240, the image buffer 250, the display system 260, and/or the archive 270.

For example, the user input device 230 may include button(s), rotary encoder(s), a touchscreen, motion tracking, voice recognition, a mouse device, keyboard, camera and/or any other device capable of receiving user directive(s). In certain embodiments, one or more of the user input devices 230 may be integrated into other components, such as the display system 260 or the ultrasound probe 204, for example.

As an example, user input device 230 may include a touchscreen display. As another example, user input device 230 may include an accelerometer, gyroscope, and/or magnetometer attached to and/or integrated with the probe 204 to provide gesture motion recognition of the probe 204, such as to identify one or more probe compressions against a patient body, a pre-defined probe movement or tilt operation, or the like. In some instances, the user input device 230 may include, additionally or alternatively, image analysis processing to identify probe gestures by analyzing acquired image data. In accordance with the present disclosure, the user input and functions related thereto may be configured to support use of new data storage scheme, as described in this disclosure. For example, the user input device 230 may be configured to support receiving user input directed at triggering and managing (where needed) application of separation process, as described herein, and/or to provide or set parameters used in performing such process. Similarly, the user input device 230 may be configured to support receiving user input directed at triggering and managing (where needed) application of the recovery process, as described herein, and/or to provide or set parameters used in performing such process.

The signal processor 240 may comprise suitable circuitry, interfaces, logic, and/or code that may be operable to process ultrasound scan data (i.e., summed IQ signal) for generating ultrasound images for presentation on a display system 260. The signal processor 240 is operable to perform one or more processing operations according to a plurality of selectable ultrasound modalities on the acquired ultrasound scan data. In an example embodiment, the signal processor 240 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 226 during a scanning session and processed in less than real-time in a live or off-line operation. In various embodiments, the processed image data can be presented at the display system 260 and/or may be stored at the archive 270.

The archive 270 may be a local archive, a Picture Archiving and Communication System (PACS), or any suitable device for storing images and related information, or may be coupled to such device or system for facilitating the storage and/or achieving of the imaging related data. In an example implementation, the archive 270 is further coupled to a remote system such as a radiology department information system, hospital information system, and/or to an internal or external network (not shown) to allow operators at different locations to supply commands and parameters and/or gain access to the image data.

The signal processor 240 may be one or more central processing units, microprocessors, microcontrollers, and/or the like. The signal processor 240 may be an integrated component, or may be distributed across various locations, for example. The signal processor 240 may be configured for receiving input information from the user input device 230 and/or the archive 270, generating an output displayable by the display system 260, and manipulating the output in response to input information from the user input device 230, among other things. The signal processor 240 may be capable of executing any of the method(s) and/or set(s) of instructions discussed herein in accordance with the various embodiments, for example.

The ultrasound imaging system 200 may be operable to continuously acquire ultrasound scan data at a frame rate that is suitable for the imaging situation in question. Typical frame rates range from 20-220 but may be lower or higher. The acquired ultrasound scan data may be displayed on the display system 260 at a display-rate that can be the same as the frame rate, or slower or faster. The image buffer 250 is included for storing processed frames of acquired ultrasound scan data that are not scheduled to be displayed immediately. Preferably, the image buffer 250 is of sufficient capacity to store at least several minutes' worth of frames of ultrasound scan data. The frames of ultrasound scan data are stored in a manner to facilitate retrieval thereof according to its order or time of acquisition. The image buffer 250 may be embodied as any known data storage medium.

In an example embodiment, the signal processor 240 may comprise a data management module 242, which comprises suitable circuitry, interfaces, logic, and/or code that may be configured to perform and/or support various functions or operations relating to, or in support of new data storage and management scheme for medical imaging solutions, as described in this disclosure.

In some implementations, the signal processor 240 (and/or components thereof, such as the data management module 242) may be configured to implement and/or use artificial intelligence and/or machine learning techniques to enhance and/or optimize imaging related functions or operations. For example, the signal processor 240 (and/or components thereof, such as the data management module 242) may be configured to implement and/or use deep learning techniques and/or algorithms, such as by use of deep neural networks (e.g., a convolutional neural network (CNN)), and/or may utilize any suitable form of artificial intelligence based processing techniques or machine learning processing functionality (e.g., for image analysis). Such artificial intelligence based image analysis may be configured to, e.g., analyze acquired ultrasound images, such as to identify, segment, label, and track structures (or tissues thereof) meeting particular criteria and/or having particular characteristics.

In an example implementation, the signal processor 240 (and/or components thereof, such as the data management module 242) may be provided as a deep neural network, which may be made up of, for example, an input layer, an output layer, and one or more hidden layers in between the input and output layers. Each of the layers may be made up of a plurality of processing nodes that may be referred to as neurons.

For example, the deep neural network may include an input layer having a neuron for each pixel or a group of pixels from a scan plane of an anatomical structure, and the output layer may have a neuron corresponding to a plurality of pre-defined structures or types of structures (or tissue(s) therein). Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The neurons of a fourth layer may learn characteristics of particular tissue types present in particular structures, etc. Thus, the processing performed by the deep neural network (e.g., convolutional neural network (CNN)) may allow for identifying biological and/or artificial structures in ultrasound image data with a high degree of probability.

In some implementations, the signal processor 240 (and/or components thereof, such as the data management module 242) may be configured to perform or otherwise control at least some of the functions performed thereby based on a user instruction via the user input device 230. As an example, a user may provide a voice command, probe gesture, button depression, or the like to issue a particular instruction, such as to initiate and/or control various aspects of the new data management scheme, including artificial intelligence (AI) based operations, and/or to provide or otherwise specify various parameters or settings relating thereto, as described in this disclosure.

The training engine 280 may comprise suitable circuitry, interfaces, logic, and/or code that may be operable to train the neurons of the deep neural network(s) of the signal processor 240 (and/or components thereof, such as the data management module 242). For example, the signal processor 240 may be trained to identify particular structures and/or tissues (or types thereof) provided in an ultrasound scan plane, with the training engine 280 training the deep neural network(s) thereof to perform some of the required functions, such as using databases(s) of classified ultrasound images of various structures.

As an example, the training engine 280 may be configured to utilize ultrasound images of particular structures to train the signal processor 240 (and/or components thereof, such as the data management module 242) with respect to the characteristics of the particular structure(s), such as the appearance of structure edges, the appearance of structure shapes based on the edges, the positions of the shapes relative to landmarks in the ultrasound image data, and the like, and/or with respect to characteristics of particular tissues (e.g., softness thereof). In various embodiments, the databases of training images may be stored in the archive 270 or any suitable data storage medium. In certain embodiments, the training engine 280 and/or training image databases may be external system(s) communicatively coupled via a wired or wireless connection to the ultrasound imaging system 200.

In operation, the ultrasound imaging system 200 may be used in generating ultrasonic images, including two-dimensional (2D), three-dimensional (3D), and/or four-dimensional (4D) images. In this regard, the ultrasound imaging system 200 may be operable to continuously acquire ultrasound scan data at a particular frame rate, which may be suitable for the imaging situation in question. For example, frame rates may range from 30-70 but may be lower or higher. The acquired ultrasound scan data may be displayed on the display system 260 at a display-rate that can be the same as the frame rate, or slower or faster. An image buffer 250 is included for storing processed frames of acquired ultrasound scan data not scheduled to be displayed immediately. Preferably, the image buffer 250 is of sufficient capacity to store at least several seconds' worth of frames of ultrasound scan data. The frames of ultrasound scan data are stored in a manner to facilitate retrieval thereof according to its order or time of acquisition. The image buffer 250 may be embodied as any known data storage medium.

In some instances, the ultrasound imaging system 200 may be configured to support grayscale and color based operations. For example, the signal processor 240 may be operable to perform grayscale B-mode processing and/or color processing. The grayscale B-mode processing may comprise processing B-mode RF signal data or IQ data pairs. For example, the grayscale B-mode processing may enable forming an envelope of the beam-summed receive signal by computing the quantity (I²+Q²)^{1/2}. The envelope can undergo additional B-mode processing, such as logarithmic compression to form the display data.

The display data may be converted to X-Y format for video display. The scan-converted frames can be mapped to grayscale for display. The B-mode frames that are provided to the image buffer 250 and/or the display system 260. The color processing may comprise processing color based RF signal data or IQ data pairs to form frames to overlay on B-mode frames that are provided to the image buffer 250 and/or the display system 260. The grayscale and/or color processing may be adaptively adjusted based on user input—e.g., a selection from the user input device 230, for example, for enhance of grayscale and/or color of particular area.

In some instances, ultrasound imaging may include generation and/or display of volumetric ultrasound images—that is where objects (e.g., organs, tissues, etc.) are displayed three-dimensional 3D. In this regard, with 3D (and similarly 4D) imaging, volumetric ultrasound datasets may be acquired, comprising voxels that correspond to the imaged objects. This may be done, e.g., by transmitting the sound waves at different angles rather than simply transmitting them in one direction (e.g., straight down), and then capture their reflections back. The returning echoes (of transmissions at different angles) are then captured, and processed (e.g., via the signal processor 240) to generate the corresponding volumetric datasets, which may in turn be used in creating and/or displaying volume (e.g. 3D) images, such as via the display 250. This may entail use of particular handling techniques to provide the desired 3D perception.

For example, volume rendering techniques may be used in displaying projections (e.g., 3D projections) of the volumetric (e.g., 3D) datasets. In this regard, rendering a 3D projection of a 3D dataset may comprise setting or defining a perception angle in space relative to the object being displayed, and then defining or computing necessary information (e.g., opacity and color) for every voxel in the dataset. This may be done, for example, using suitable transfer functions for defining RGBA (red, green, blue, and alpha) value for every voxel.

In some embodiments, the ultrasound imaging system 200 may be configured to support implementing and using digital imaging and communications in medicine (DICOM) structured reporting (SR) object consolidation in accordance with the present disclosure. In this regard, as described in this disclosure medical imaging systems and/or environments may be configured to support implementing and using enhanced solutions for storage and management of medical imaging data, particularly in facilitating consolidating multiple SR objects based on image files, as described with respect to FIG. 1 and illustrated in the example use case scenario shown and described with respect to FIG. 4.

For example, once the imaging data is obtained or generated, the signal processor 240 (and/or components thereof, such as the data management module 242) may store the processed image files in the archive 270, which may be configured to apply, independently or under control of signal processor 240 (and/or components thereof, such as the data management module 242), archiving based encoding (e.g., DICOM based encoding) to the data, and then perform storage and management related functions (e.g., based on the DICOM standard), including performing required communication functions for transmitting the resultant encoded data objects to corresponding storage locations (local or remote).

The archive 270 may also be configured to retrieve the encoded data back, and as such may be configured to perform a recovery process. In this regard, the archive 270 may be configured to apply the recovery process to previously archived data, including performing any required communication functions, for requesting and receiving data files from storage locations (local or remote), and to decode the data to enable generating corresponding images, such as for display via the display system 260. These functions may be controlled or managed by the signal processor 240 (and/or components thereof, such as the data management module 242). Alternatively, the archive 270 may also be configured to perform at least some of these functions independently, and as such the processor 240 may not be even know that the data underwent any separation.

Further, the ultrasound imaging system 200 (e.g., particularly via the processor 240, and/or components thereof, such as the data management module 242) may be configured to handle multiple SR objects, particularly in accordance with a consolidation scheme/methodology in accordance with the present disclosure may be used for handling the consolidation of the SR objects. In this regard, as noted in some instances image files, generated based on ultrasound imaging, may be processed based on the DICOM standard for storage, management and/or communication thereof. This may result in corresponding SR objects as these image files are studied and/or analyzed. This may result in multiple SR objects, and as such the consolidation scheme/methodology may be used for handling the consolidation of the SR objects as described herein. An example use case scenario with multiple SR objects and handling thereof is described in more detail with respect to FIG. 4.

In some instances, at least a portion of the consolidation scheme/methodology may be performed within the ultrasound imaging system 200, particularly via the processor 240 (and/or components thereof, such as the data management module 242), which may be configured to run applications that process or handle DICOM SR objects. Alternatively or additionally, at least a portion of the consolidation scheme/methodology may be offloaded to an external system (e.g., an instance of the computer system 120 as described with respect to FIG. 1).

Further, in some instances, the consolidation scheme/methodology, and implementation or performing thereof, may entail use of advance processing techniques, such as artificial intelligence (AI) or other machine learning techniques. In this regard, the ultrasound imaging system 200, particularly via the processor 240 (and/or components thereof, such as the data management module 242) may be configured to implement and/or support use of artificial intelligence (AI) based learning mode in conjunction with the consolidation scheme/methodology. For example, the data management module 242 (and the training engine 280) may be configured to support and use artificial intelligence (AI) based learning mode when running or using the consolidation scheme/methodology, such as to recognize anomalies and/or to automatically make common manual anomaly reconciliations. Alternatively or additionally, at least a portion of the artificial intelligence (AI) based learning mode related functions may be offloaded to an external system (e.g., local dedicated computing system, remote (e.g., Cloud-based) server, etc.).

The ultrasound imaging system 200 may also be configured to support use and handling of composite SR objects that may result from the consolidation scheme/methodology, as described herein. For example, the archive 270 may be configured to handle such composite SR objects when applying the recovery process as described above.

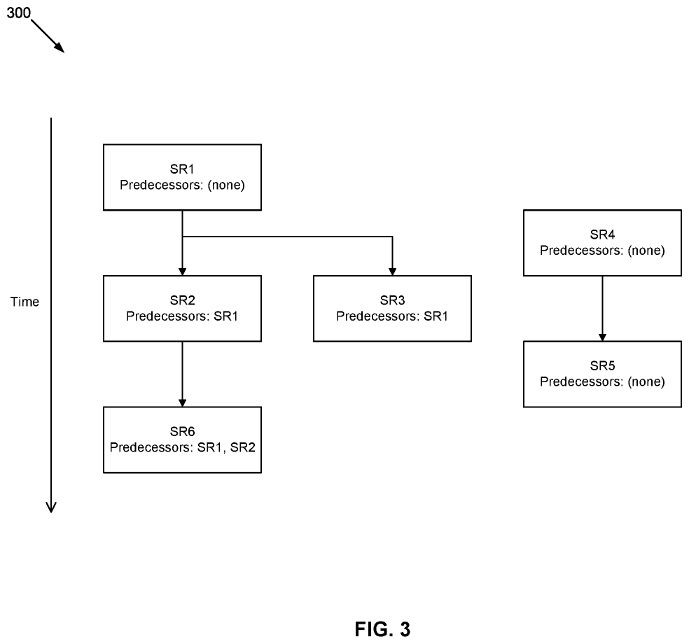

FIG. 3 is a block diagram illustrating an example use case scenario with consolidation of multiple digital imaging and communications in medicine (DICOM) structured reporting (SR) objects. Shown in FIG. 3 is diagram 300 depicting relations among and handling of a plurality of DICOM SR objects (SR1-SR6) with consolidation.

In this regard, as described above, DICOM SR objects may store information associated with image files, such as findings from a medical procedure (study), which may include measurements, calculations, interpretations, etc. SR objects may typically include two mandatory state tags: “Completion Flag” and “Verification Flag”. The “Completion Flag” may have a value of “COMPLETE” or “PARTIAL.” The “Verification Flag” may have a value of “VERIFIED” or “UNVERIFIED”. These tags may be used to convey relevant content related information, such as responsibility for content completion. SR objects that are “COMPLETE” and “VERIFIED” may be used as a “source of truth” (e.g., for the findings in the study). However, when there are only “PARTIAL” SR objects, consumers of the SR object sets may have difficulty consolidating discrete data elements and resolving conflicts. In this regard, a “data element” may refer to both the label and the value, thus there may be multiple data elements with the same label. The label includes optional codified “modifiers” that describe the context of the value. The difficulty in consolidating SR objects may be especially true for particular types of studies, such as Echocardiographic Ultrasound (Echo) studies, which employ many measurements and calculations across multiple measurement sessions.

In an example cardio-based use scenario/study, SR objects may be used to store findings relating to cardiovascular orifice area of the aortic valve. In this regard, the cardiovascular orifice area of the aortic valve may have the following encoding: measurement type: cardiovascular orifice area; site: aortic valve; image mode: 2D; measurement method: planimetry; direction of flow: antegrade flow; value: 1.391677163142; units: square centimeters (cm²). Such encoding is not mandatory/fixed, however, and as such another party (e.g., another vendor) may encode the cardiovascular orifice area of the aortic valve differently—e.g., skipping measurement method and adding selection status: mean value chosen. DICOM SR objects have “Templates” defined by the DICOM 3.0 Standard for various clinical use cases e.g., “Adult Echocardiography.”

Individual SR objects in a study may be independent or an aggregation of previous DICOM SR objects. For instances where aggregation is used, the DICOM standard defines an optional field called “Predecessor Documents Sequence” that lists the “parent” DICOM SR objects whose content was inherited into the new “child” DICOM SR object. However, when creating SR objects with the Predecessor Documents Sequence, it is still possible for them to “diverge”, for example, when two users concurrently make a new SR object from the same source SR object. Such new objects may be referred as “divergent SR objects.” A number of possible problem scenarios may be encountered when multiple SR objects exist in a study (the same study), however. The following table lists possible problem scenarios in such a study.

TABLE 1
possible problem scenarios when consolidating multiple SR objects

| | Challenges Consuming SR | Example SR |
|---|---|---|
| Scenarios | Object | Object |
| 1. Individual SR object with | Accumulating values from | SR4 |
| unique findings so no Predecessor | multiple SR objects | |
| Documents Sequence | | |
| 2. SR object with values inherited | Avoid duplicating a value found | SR5 |
| from previous SR object but no | in parent and child SR object. | |
| Predecessor Documents Sequence | Excluding values deleted from | |
| | child SR object. | |
| 3. SR object with values inherited | Avoid duplicating a value found | SR1, SR2, and |
| from previous SR object with | in parent and child SR object. | SR6 |
| Predecessor Documents Sequence | Excluding values deleted from | |
| | child SR object. | |
| 4. Multiple SR objects containing a | Avoid duplicating a value found | SR3 |
| Predecessor Documents Sequence, | in child SR objects. | |
| but they “diverge” creating more | Recognizing values added or | |
| than one child SR object | deleted from child SR objects. | |

In accordance with the present disclosure, a consolidation scheme/methodology may be used to handle the consolidation of the multiple SR objects. In this regard, the consolidation scheme/methodology described herein may be applies adaptively, such as only to SR objects with Completion Flag of “PARTIAL” and the same Template. The following consolidation scheme/methodology may be used to process the various possible problem scenarios detailed in the table above into a Composite SR object: 1) inspect the Predecessor Documents Sequence of the SR objects discarding any SR objects that are a parent of another SR object. In this regard, as used herein, “discarding” SR objects does not necessarily entail deleting such objects; rather, these objects may simply ignored without actually being deleted. This may be particularly the case with DICOM objects as it is common practice to avoid deleting objects from an archive since such objects are part of the permanent medical record. If only one SR object remains, then copy it to make the Consolidated SR object; 2) otherwise, for each SR object that remains, process them from newest to oldest, using the Content Date and Content Time fields for sorting; and 3) for each SR object, copy each data element found into the Composite SR object, and remove it from any of the unprocessed SR objects to avoid copying it again later; copy any duplicate findings that exist in a single SR object because there is clinical value in knowing an identical finding was created twice.

The consolidation scheme results in creating a new Composite SR object every time a new DICOM SR object is added to a study. In this way, a Composite SR object may always exist in the Study. All consumers of DICOM SR objects will be able to easily find the correct and unique Composite SR by inspecting the Predecessor Documents Sequences.

For example, the diagram 300 of FIG. 3 illustrates an example use case with all 4 problem scenarios described in the table above present. The consolidation scheme will process the SR object set in the following steps: 1) discard SR1 and SR2; 2) process SR6; 3) process SR5; 4) process SR3 (divergence case, SR3 and SR2 are divergent from one another); 5) process SR4 (predecessor tag was not saved in SR5); and 6) create new Composite SR object listing all 6 SR objects as predecessors so they can be ignored by SR consumers.

In some instances, object consideration may entail encountering and handling special use cases. For example, in a special use case deleted data elements may exist and may need to be addressed. In this regard, when the Predecessor Documents Sequence is present, deletions may be handled automatically because they will not exist in the child SR object and the parent SR objects will be ignored. However, when the Predecessor Documents Sequence is not present, it is possible to encounter an older SR object containing a data element that was deleted in a newer SR object. There is no way to differentiate a true deletion from the case where SR objects came from different sources, with content added by both sources.

Accordingly, measures for handling such conditions may be used. For example, the following options may be used to handle these potential deletions: 1) provide a configuration to control if possible deletions should be retained or not; 2) provide a reconciliation tool for an administrator to resolve possible deletions; and 3) the reconciliation tool may incorporate a learning mechanism (e.g., an AI “learn mode”) to recognize common reconciliation patterns (e.g., retain or remove) for specific findings allowing it to be done automatically.

In another special use case users may only want the newest instance of a finding to be retained. In this regard, when the Predecessor Documents Sequence is present, the old finding will be ignored because the old SR object will be discarded. However, when the Predecessor Documents Sequence is not present, measures for handling such conditions may be used. For example, the following options may be used to handle multiple instances of a finding: 1) provide a configuration to control if multiple instances of a finding should be retained or only the newest instance; 2) if present, make use of an optional qualifier indicating how to handle multiple finding instances, e.g., Maximum, Minimum, First, Last, Average; 3) provide a reconciliation tool for an administrator to manage multiple instances of a finding; and 4) the reconciliation tool may incorporate a learning mechanism (e.g., an AI “learn mode”) to recognize common reconciliation patterns (keep all or keep last) for specific findings allowing it to be done automatically.

In another special use case understanding semantics of findings may be necessary for the execution of the methodology. In this regard, it is possible for 2 SR objects to represent the same finding instance with a different set of modifiers. The methodology may be configured to employ semantic interpretation to allow for recognizing these findings as the same instance. However, the methodology still works without semantic interpretation, with the possibility of replication of some finding instances. Additionally, a learning mechanism (e.g., an AI “learn mode”) may be used and configured to detect patterns for findings with identical values but slightly different representations per different vendor, allowing for automatically detecting the findings as duplicates.

The AI “learn mode” may be implemented in and/or provided by suitable components of the system, such as the signal processor 240 (and particularly components thereof, such as the data management module 242, in conjunction with the training engine 280) in the ultrasound system 200.

In an example implementation, an audit log may be maintained to track actions taken by an administrator or by an AI mode for the special cases above.

FIG. 4 illustrates a flowchart of an example process for digital imaging and communications in medicine (DICOM) structured reporting (SR) object consolidation. Shown in FIG. 4 is flow chart 400, comprising a plurality of example steps (represented as blocks 402-416), which may be performed in a suitable system (e.g., the medical imaging system 110 of FIG. 1, or the ultrasound imaging system 200 of FIG. 2) for digital imaging and communications in medicine (DICOM) structured reporting (SR) object consolidation.

In start step 402, the system may be setup, and operations may initiate.

In step 404, imaging signals may be obtained during a medical imaging based examination. This may be done by transmitting certain types of signals, and then capturing echoes of these signals. For example, in an ultrasound imaging system (e.g., the ultrasound system 200 of FIG. 2), this may comprise transmitting ultrasound signals and receiving corresponding echoes of the ultrasound signals.

In step 406, the imaging signals (e.g., the received echoes of the ultrasound signals) may be processed (e.g., via the display/control unit 114 of the medical imaging system 110, or the signal processor 240 of the ultrasound system 200), to generate corresponding imaging data for use in generating corresponding medical images (e.g., ultrasound images). In some instances, at least a portion of the data generation may be performed in a different system than the one where the imaging signals are captured.

In step 408, the generated image data (e.g., image files) may be processed for archiving, particularly in accordance with a particular standard such as DICOM. This may be comprise applying encoding (e.g., DICOM based encoding) to the image data. In some instances, at least a portion of the archiving may be performed in a different system than the one where the imaging signals are captured and/or the image data is generated.

In step 410, a plurality of objects (e.g., DICOM SR objects) may be generated based on the imaging data—e.g., by multiple users and/or multiple runs/assessments (including a same user).

When such a plurality of objects is generated, object consolidation may be performed. This starts in step 412, each object in the plurality of DICOM SR objects may be assessed. The assessing may comprise determining whether the object is a parent of another object in the plurality of objects; and when the object is determined to be a parent of another object, the object getting discarded. In step 414, a check may be performed to determine whether all objects had been assessed, with the process proceeding to step 414 when all objects have been processed (i.e., “yes” condition), and looping back to step 412 when not all objects are processed (i.e., “no” condition).

In step 416, a composite object (e.g., DICOM SR composite object) may be generated. In this regard, generating the composite object may comprise, when only one object remains after the assessing, copying the one object into the consolidated object; when a plurality of remaining objects remains after the assessing, the plurality of remaining objects is processed, with the processing performed in sequence from newest to oldest, and with the processing comprising, for each remaining object, copying each data element found into the composite object; and removing the remaining object (from processing list).

An example method for managing medical data, in accordance with the present disclosure, comprises applying, by a processor, a consolidation process for consolidating a plurality of objects, wherein the plurality of objects is generated based on a same medical imaging data; and wherein the consolidation process comprises: assessing each object of the plurality of objects, wherein the assessing comprises: determining whether the object is a parent of another object in the plurality of objects; and when the object is a parent of another object, discarding the object; and generating a composite object based on the plurality of objects, wherein the generating comprises: when only one object remains after the assessing, copying the one object into the consolidated object; and when a plurality of remaining objects remains after the assessing, processing the plurality of remaining objects, wherein the processing is performed in sequence from newest to oldest, and wherein the processing comprises, for each remaining object: copying each data element found into the composite object; and discarding the remaining object.

In an example embodiment, the medical dataset comprises Digital Imaging and Communications in Medicine (DICOM) based dataset.

In an example embodiment, each of the plurality of objects comprises Digital Imaging and Communications in Medicine (DICOM) structured reporting (SR) object.

In an example embodiment, the method further comprises sorting remaining objects in a plurality of remaining objects from newest to oldest based on DICOM SR object based content date and content time fields.

In an example embodiment, assessing the object comprises determining a Predecessor Unique Identifier (UID) Sequence of the object, and determining when the object is a parent of another object based on matching of the Predecessor Documents Sequence.

In an example embodiment, processing the plurality of remaining objects further comprises copying any duplicate finding that exists in a single object.

In an example embodiment, the method further comprises processing the plurality of remaining objects further comprises discarding any finding that is duplicated of another remaining object already processed.

In an example embodiment, the method further comprises utilizing artificial intelligence when applying the consolidation process.

In an example embodiment, the method further comprises applying artificial intelligence based learning for recognizing common reconciliation patterns in findings during the processing of the plurality of remaining objects.

In an example embodiment, the method further comprises configuring at least a portion of the consolidation process based on user input.

In an example embodiment, the method further comprises maintaining an audit log, wherein the audit log comprises data from tracking actions taken in conjunction with the consolidating of the plurality of objects.

An example system for managing medical data, in accordance with the present disclosure, comprises at least one processing circuit configured to apply a consolidation process for consolidating a plurality of objects, with the plurality of objects is generated based on a same medical imaging data. The at least one processing circuit is configured to, when applying the consolidation process: assess each object of the plurality of objects, with the assessing comprising: determining whether the object is a parent of another object in the plurality of objects, and when the object is a parent of another object, discarding the object, and generating a composite object based on the plurality of objects, with the generating comprising: when only one object remains after the assessing, copying the one object into the consolidated object, and when a plurality of remaining objects remains after the assessing, processing the plurality of remaining objects, wherein the processing is performed in sequence from newest to oldest, and wherein the processing comprises, for each remaining object: copying each data element found into the composite object, and discarding the remaining object.

In an example embodiment, each of the plurality of objects comprises Digital Imaging and Communications in Medicine (DICOM) structured reporting (SR) object, and the at least one processing circuit is configured to sort remaining objects in a plurality of remaining objects from newest to oldest based on DICOM SR object based content date and content time fields.

In an example embodiment, each of the plurality of objects comprises Digital Imaging and Communications in Medicine (DICOM) structured reporting (SR) object, and the at least one processing circuit is configured to, when assessing the object: determine DICOM based Predecessor Unique Identifier (UID) Sequence of the object, and determine when the object is a parent of another object based on matching of the Predecessor Documents Sequence.

In an example embodiment, the at least one processing circuit is configured to, when processing the plurality of remaining objects, copy any duplicate finding that exists in a single object.

In an example embodiment, the at least one processing circuit is configured to, when processing the plurality of remaining objects, discard any finding that is duplicated of another remaining object already processed.

In an example embodiment, the at least one processing circuit is configured to utilize artificial intelligence when applying the consolidation process.

In an example embodiment, the at least one processing circuit is configured to utilize and/or apply artificial intelligence based learning for recognizing common reconciliation patterns in findings during the processing of the plurality of remaining objects.

In an example embodiment, the at least one processing circuit is configured to configure or adjust at least a portion of the consolidation process based on user input.

In an example embodiment, the at least one processing circuit is configured to maintain an audit log, wherein the audit log comprises data from tracking actions taken in conjunction with the consolidating of the plurality of objects.

As utilized herein the terms “circuits” and “circuitry” refer to physical electronic components (e.g., hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.” As utilized herein, the terms “block” and “module” refer to functions than can be performed by one or more circuits. As utilized herein, the term “exemplary” means serving as a non-limiting example, instance, or illustration. As utilized herein, the terms “for example” and “e.g.,” set off lists of one or more non-limiting examples, instances, or illustrations. As utilized herein, circuitry is “operable” to perform a function whenever the circuitry comprises the necessary hardware (and code, if any is necessary) to perform the function, regardless of whether performance of the function is disabled or not enabled (e.g., by some user-configurable setting, a factory trim, etc.).

Other embodiments of the invention may provide a non-transitory computer readable medium and/or storage medium, and/or a non-transitory machine readable medium and/or storage medium, having stored thereon, a machine code and/or a computer program having at least one code section executable by a machine and/or a computer, thereby causing the machine and/or computer to perform the processes as described herein.

Accordingly, the present disclosure may be realized in hardware, software, or a combination of hardware and software. The present invention may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may be a general-purpose computing system with a program or other code that, when being loaded and executed, controls the computing system such that it carries out the methods described herein. Another typical implementation may comprise an application specific integrated circuit or chip.

Various embodiments in accordance with the present disclosure may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A method for managing medical data, the method comprising:
applying, by a processor, a consolidation process for consolidating a plurality of objects,
wherein the plurality of objects is generated based on a same medical imaging data; and
wherein the consolidation process comprises:
assessing each object of the plurality of objects, wherein the assessing comprises:
determining whether the object is a parent of another object in the plurality of objects; and
when the object is a parent of another object, discarding the object; and
generating a composite object based on the plurality of objects, wherein the generating comprises:
when only one object remains after the assessing, copying the one object into the consolidated object; and
when a plurality of remaining objects remains after the assessing, processing the plurality of remaining objects,
wherein the processing is performed in sequence from newest to oldest, and
wherein the processing comprises, for each remaining object:
copying each data element found into the composite object; and
discarding the remaining object.

2. The method of claim 1, wherein the medical dataset comprises Digital Imaging and Communications in Medicine (DICOM) based dataset.

3. The method of claim 1, wherein each of the plurality of objects comprises Digital Imaging and Communications in Medicine (DICOM) structured reporting (SR) object.

4. The method of claim 3, further comprising sorting remaining objects in a plurality of remaining objects from newest to oldest based on DICOM SR object based content date and content time fields.

5. The method of claim 3, wherein assessing the object comprises determining a Predecessor Unique Identifier (UID) Sequence of the object, and determining when the object is a parent of another object based on matching of the Predecessor Documents Sequence.

6. The method of claim 1, wherein processing the plurality of remaining objects further comprises copying any duplicate finding that exists in a single object.

7. The method of claim 1, wherein processing the plurality of remaining objects further comprises discarding any finding that is duplicated of another remaining object already processed.

8. The method of claim 1, further comprising utilizing artificial intelligence when applying the consolidation process.

9. The method of claim 8, further comprising applying artificial intelligence based learning for recognizing common reconciliation patterns in findings during the processing of the plurality of remaining objects.

10. The method of claim 1, further comprising configuring at least a portion of the consolidation process based on user input.

11. The method of claim 1, further comprising maintaining an audit log, wherein the audit log comprises data from tracking actions taken in conjunction with the consolidating of the plurality of objects.

12. A system for managing medical data, the system comprising:
at least one processing circuit configured to apply a consolidation process for consolidating a plurality of objects,
wherein the plurality of objects is generated based on a same medical imaging data; and
wherein the at least one processing circuit is configured to, when apply the consolidation process:
assess each object of the plurality of objects, wherein the assessing comprises:
determining whether the object is a parent of another object in the plurality of objects; and
when the object is a parent of another object, discarding the object; and
generate a composite object based on the plurality of objects, wherein the generating comprises:
when only one object remains after the assessing, copying the one object into the consolidated object; and
when a plurality of remaining objects remains after the assessing, processing the plurality of remaining objects,
wherein the processing is performed in sequence from newest to oldest, and
wherein the processing comprises, for each remaining object:
copying each data element found into the composite object; and
discarding the remaining object.

13. The system of claim 12, wherein each of the plurality of objects comprises Digital Imaging and Communications in Medicine (DICOM) structured reporting (SR) object, and
wherein the at least one processing circuit is configured to sort remaining objects in a plurality of remaining objects from newest to oldest based on DICOM SR object based content date and content time fields.

14. The system of claim 12, wherein each of the plurality of objects comprises Digital Imaging and Communications in Medicine (DICOM) structured reporting (SR) object, and
wherein the at least one processing circuit is configured to, when assessing the object:
determine DICOM based Predecessor Unique Identifier (UID) Sequence of the object, and
determine when the object is a parent of another object based on matching of the Predecessor Documents Sequence.

15. The system of claim 12, wherein the at least one processing circuit is configured to, when processing the plurality of remaining objects, copy any duplicate finding that exists in a single object.

16. The system of claim 12, wherein the at least one processing circuit is configured to, when processing the plurality of remaining objects, discard any finding that is duplicated of another remaining object already processed.

17. The system of claim 12, wherein the at least one processing circuit is configured to utilize artificial intelligence when applying the consolidation process.

18. The system of claim 17, wherein the at least one processing circuit is configured to utilize and/or apply artificial intelligence based learning for recognizing common reconciliation patterns in findings during the processing of the plurality of remaining objects.

19. The system of claim 12, wherein the at least one processing circuit is configured to configure or adjust at least a portion of the consolidation process based on user input.

20. The system of claim 12, wherein the at least one processing circuit is configured to maintain an audit log, wherein the audit log comprises data from tracking actions taken in conjunction with the consolidating of the plurality of objects.
